(19) United States
(12) Patent Application Publication
(10) Pub. No.: US 2012/0302526 A1
(43) Pub. Date: Nov. 29, 2012

(54) METHODS FOR TREATING POST-TRAUMATIC STRESS DISORDER

(75) Inventors: Daniel Stephen Barth, Boulder, CO (US); Krista Marie Rodgers, Boulder, CO (US)

(73) Assignee: Regents of the University of Colorado, a body corporate

(21) Appl. No.: 13/295,798

(22) Filed: Nov. 14, 2011

Related U.S. Application Data
(60) Provisional application No. 61/491,106, filed on May 27, 2011.

(51) Int. Cl. A61K 31/437; A61K 31/522; A61P 25/28; A61K 31/65; A61P 25/00; A61P 25/22

(52) U.S. Cl. 514/154; 514/300; 514/263.32

(57) ABSTRACT

Provided herein are methods for treating negative effects of traumatic brain injury (TBI), post traumatic stress disorder (PTSD), or acute stress disorder (ASD). These methods comprise administration of an effective amount of ibudilast to a subject suffering from TBI, PTSD, or ASD.

### CROSS-REFERENCE TO RELATED APPLICATION

This application claims priority to U.S. provisional application No. 61/491,106 filed on May 27, 2011, which is incorporated herein in its entirety by reference.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant No. 1 R01 NS36981 awarded by National Institute of Neurological Disorders and Stroke.

### FIELD OF THE INVENTION

The present invention relates to methods for treating post-traumatic stress disorder, acute stress disorder, and negative effects of each thereof and negative effects of traumatic brain injury, in particular by administering ibudilast.

### BACKGROUND OF THE INVENTION

Post-Traumatic Stress Disorder (PTSD) and acute stress disorder (ASD) are anxiety disorder that can develop after exposure to a terrifying event or ordeal in which grave physical harm occurred or was threatened. Traumatic events that trigger PTSD or ASD include traumatic brain injury (TBI). TBI itself can lead to a variety of anxiety disorders. It is estimated that the lifetime prevalence of PTSD in the U.S. is approximately 8% of the U.S. population. The rate among former combat soldiers runs much higher.

### SUMMARY OF THE INVENTION

In one aspect, the present invention provides a method of alleviating one or more negative effects of anxiety exhibited by a subject suffering from a traumatic brain injury (TBI) comprising administering to the subject in need thereof an effective amount of ibudilast or a pharmaceutically acceptable salt thereof. In another aspect, the present invention provides a method of inhibiting an onset of one or more negative effects of TBI exhibited by a subject suffering from TBI comprising administering to the subject in need thereof an effective amount of ibudilast or a pharmaceutically acceptable salt thereof. In another aspect, the present invention provides a method of alleviating one or more negative effects of anxiety exhibited by a subject suffering from a TBI comprising administering pre-injury to the subject who is at risk for TBI an effective amount of ibudilast or a pharmaceutically acceptable salt thereof. In another aspect, the present invention provides a method of attenuation of glial cell activation in a subject suffering from TBI comprising administering to the subject in need thereof an effective amount of ibudilast or a pharmaceutically acceptable salt thereof.

In another aspect, the present invention provides a method of preventing or reducing a frequency of occurrence of one or more negative effects of post traumatic stress disorder (PTSD) or acute stress disorder (ASD) in a subject suffering from a PTSD or an ASD comprising administering to the subject in need thereof an effective amount of ibudilast or a pharmaceutically acceptable salt thereof. In another aspect, the present invention provides a method of alleviating one or more negative effects of PTSD or ASD in a subject suffering from a PTSD or an ASD comprising administering to the subject in need thereof an effective amount of ibudilast or a pharmaceutically acceptable salt thereof. In another aspect, the present invention provides a method of alleviating one or more negative effects of PTSD or ASD in an individual who is on active duty in a branch of a military or who is a military veteran comprising administering to the individual in need thereof an effect amount of ibudilast or a pharmaceutically acceptable salt thereof.

In another aspect, the present invention provides a method for treating PTSD or ASD in a subject comprising administering to the subject in need thereof an effective amount of a TLR-4 antagonist. In another aspect, the present invention provides a method for preventing or reducing the frequency of post-traumatic stress disorder occurrence in a subject comprising administering to the subject in need thereof a therapeutically effective amount of a TLR-4 antagonist. In one embodiment, the TLR-4 antagonist is ibudilast or a pharmaceutically acceptable salt thereof.

In another embodiment, the ibudilast or the pharmaceutically acceptable salt thereof is administered in an amount ranging from about 30 to about 300 mg free base equivalent of ibudilast per day, preferably 40 to 120 mg free base equivalent of ibudilast per day in a delayed release oral pharmaceutical composition, and more preferably 80 to 100 mg free base equivalent of ibudilast per day as an oral drug product (capsule) such as Pinatos® or Ketas®.

In another embodiment, ibudilast or the pharmaceutically acceptable salt thereof is administered orally, or by subcutaneous, intravenous, or intranasal injection. In another embodiment, the ibudilast or the pharmaceutically acceptable salt thereof is administered once daily, twice daily, thrice daily, once every two days, once every three days, or once a week. In another embodiment, the ibudilast or the pharmaceutically acceptable salt thereof is administered for five days, two weeks, one month, two months, three months, six months, a year, two years, or for three or more years. In another embodiment, the method further comprises administering a glial cell attenuating drug. In another embodiment, the glial cell attenuating drug is minocycline, propentofylline, or pentoxifyline.

### BRIEF DESCRIPTION OF THE DRAWINGS

FIG. 1 graphically illustrates the efficacy of ibudilast (MN166) administered post-injury to alleviate TBI induced anxiety-like behavior in vivo.

FIG. 2 graphically illustrates the efficacy of ibudilast administered pre-injury, during injury, and post-injury to alleviate TBI induced anxiety-like behavior in vivo.

FIG. 3A, in comparison with FIG. 3B, graphically illustrates that electric shocks and novel contexts when added to LFPI, enhance anxiety-like behavior in vivo.

FIG. 3B graphically illustrates the efficacy of ibudilast administered pre-injury, during injury, and post-injury to alleviate TBI induced anxiety-like behavior in vivo.

FIG. 4A, in comparison with FIG. 4B, graphically illustrates that electric shocks when added to LFPI, enhance anxiety-like behavior in vivo.

FIG. 4B graphically illustrates the efficacy of ibudilast administered pre-injury, during injury, and post-injury to alleviate TBI induced anxiety-like behavior in vivo.

### DETAILED DESCRIPTION OF THE INVENTION

### Definitions

“Acute stress disorder (ASD)” is an anxiety disorder that involves a reaction following exposure to a traumatic event or stressor (e.g., a serious injury to oneself, witnessing an act of violence, hearing about something horrible that has happened to someone one is close to). While similar to PTSD, the duration of symptoms of ASD is shorter than that for PTSD. For a diagnosis of ASD, the full range of symptoms may be present for two days to four weeks.

“Administering” or “administration of” an agent to a subject (and grammatical equivalents of this phrase) includes both direct administration, including self-administration, and indirect administration, including the act of prescribing a drug. For example, as used herein, a physician who instructs a patient to self-administer a drug and/or provides a subject with a prescription for a drug is administering the drug to the subject.

“Antagonist” refers to a compound or a composition that attenuates the effect of an agonist. The antagonist can bind reversibly or irreversibly to a region of the receptor in common with an agonist. Antagonist can also bind at a different site on the receptor or an associated ion channel. Moreover, the term “antagonist” also includes functional antagonist or physiological antagonist. Functional antagonist refers to a compound and/or compositions that reverses the effects of an agonist rather than acting at the same receptor, i.e., functional antagonist causes a response in the tissue or animal which opposes the action of an agonist. Examples include agents which have opposing effects on an intracellular second messenger, or, in an animal, on blood pressure. A functional antagonist can sometimes produce responses which closely mimic those of the pharmacological kind.

“Ibudilast” or MN166 or AV-411 refers to a compound of formula:

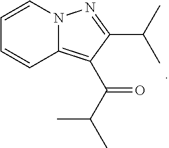

“Negative effect” refers to a symptom that affects the subject in a deleterious or negative manner.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is safe, non-toxic, and pharmaceutically acceptable, and that possesses the desired pharmacological activity of the parent compound. Such salts include acid addition salts, formed with inorganic acids such as hydrochloric acid, hydrobromic acid, sulfuric acid, nitric acid, phosphoric acid, and the like; or salts formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1 carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Post-Traumatic Stress Disorder (PTSD)” is an anxiety disorder that can develop after exposure to a terrifying event or ordeal in which grave physical harm occurred or was threatened to oneself or others. Traumatic events that may trigger PTSD include violent personal assaults, natural or human-caused disasters, accidents, or military combat, all of which can involve traumatic brain injury (TBI). PTSD was described in veterans of the American Civil War, and was called “shell shock,” “combat neurosis,” and “operational fatigue.” PTSD symptoms can be grouped into three categories: (1) re-experiencing symptoms; (2) avoidance symptoms; and (3) hyperarousal symptoms. Exemplary re-experience symptoms include flashbacks (e.g., reliving the trauma over and over, including physical symptoms like a racing heart or sweating), bad dreams, and frightening thoughts. Re-experiencing symptoms may cause problems in a person's everyday routine. They can start from the person's own thoughts and feelings. Words, objects, or situations that are reminders of the event can also trigger re-experiencing. Symptoms of avoidance include staying away from places, events, or objects that are reminders of the experience; feeling emotionally numb; feeling strong guilt, depression, or worry; losing interest in activities that were enjoyable in the past; and having trouble remembering the dangerous event. Things that remind a person of the traumatic event can trigger avoidance symptoms. These symptoms may cause a person to change his or her personal routine. For example, after a bad car accident, a person who usually drives may avoid driving or riding in a car. Hyperarousal symptoms include being easily startled, feeling tense or “on edge”, having difficulty sleeping, and/or having angry outbursts. Hyperarousal symptoms are usually constant, instead of being triggered by things that remind one of the traumatic event. They can make the person feel stressed and angry. These symptoms may make it hard to do daily tasks, such as sleeping, eating, or concentrating. Therefore, generally, PTSD symptoms can include nightmares, flashbacks, emotional detachment or numbing of feelings (emotional self-mortification or dissociation), insomnia, avoidance of reminders and extreme distress when exposed to the reminders (“triggers”), loss of appetite, irritability, hypervigilance, memory loss (may appear as difficulty paying attention), excessive startle response, clinical depression, stress, and anxiety. The symptoms may last for a month, for three months, or for longer periods of time.

“Reduction” of a symptom or symptoms (and grammatical equivalents of this phrase) refers to decreasing the severity and/or frequency of the symptom(s), and/or elimination of the symptom(s).

“Subject” refers to any mammal, such as rodents (non limiting examples of which include rats), simians, dogs, and humans.

“Therapeutically effective amount” or “effective amount” means the amount of a compound that, when administered to a subject for treating a disease, will have the intended therapeutic effect, e.g., alleviation, amelioration, palliation, reduction, or elimination of one or more symptoms or negative effects of the disease. The full therapeutic effect does not necessarily occur by administration of one dose (or dosage), and may occur only after administration of a series of doses. Thus, an effective amount may be administered in one or more administrations. The “therapeutically effective amount” will vary depending on the compound, the disease, and its severity and the age, weight, etc., of the mammal to be treated.

“Traumatic brain injury (TBI)”, is damage to the brain as the result of an injury. TBI usually results from a violent blow or jolt to the head that causes the brain to collide with the inside of the skull. An object penetrating the skull, such as a bullet or shattered piece of skull, can also cause TBI. Depending on the severity of the blow or jolt to the head, TBI can be a mild TBI or moderate to severe TBI. Mild TBI may cause temporary dysfunction of brain cells. More serious TBI can result in bruising, torn tissues, bleeding and other physical damage to the brain that can result in long-term complications. The signs and symptoms of mild TBI may include: confusion or disorientation, memory or concentration problems, headache, dizziness or loss of balance, nausea or vomiting, sensory problems, such as blurred vision, ringing in the ears or a bad taste in the mouth, sensitivity to light or sound, mood changes or mood swings, feeling depressed or anxious, fatigue or drowsiness, difficulty sleeping, or sleeping more than usual. Moderate to severe TBI can include any of the signs and symptoms of mild injury, as well as the following symptoms that may appear within the first hours to days after a head injury: profound confusion, agitation, hyperexcitability, combativeness or other unusual behavior, slurred speech, inability to awaken from sleep, weakness or numbness in the extremities, loss of coordination, persistent headache or headache that worsens, convulsions or seizures. Symptoms of TBI also include cognitive or memory impairments and motor deficits. TBI may cause negative effects such as emotional, social, or behavioral problems, changes in personality, emotional instability, depression, anxiety, hypomania, mania, apathy, irritability, problems with social judgment, and impaired conversational skills. TBI appears to predispose survivors to psychiatric disorders including obsessive compulsive disorder, substance abuse, dysthymia, clinical depression, bipolar disorder, and anxiety disorders. In patients who have depression after TBI, suicidal ideation is common; the suicide rate among these patients increase 2- to 3-fold. Social and behavioral effects that can follow TBI include disinhibition, inability to control anger, impulsiveness, and lack of initiative.

“Treating” or “treatment” of a disease includes: (1) preventing or prophylaxis of the disease, i.e., causing the clinical symptoms or the negative effects of the disease not to develop in a subject that may be exposed to or predisposed to the disease but does not yet experience or display symptoms of the disease; (2) inhibiting the disease, i.e., alleviating, arresting, or reducing the development of the disease, or its clinical symptoms or negative effects; or (3) relieving the disease, i.e., causing regression of the disease, or its clinical symptoms or negative effects.

### PREFERRED EMBODIMENTS

In one aspect, the present invention provides a method of alleviating one or more negative effects of anxiety exhibited by a subject suffering from a traumatic brain injury (TBI) comprising administering to the subject in need thereof an effective amount of ibudilast or a pharmaceutically acceptable salt thereof. In another aspect, the present invention provides a method of inhibiting an onset of one or more negative effects of TBI exhibited by a subject suffering from TBI comprising administering to the subject in need thereof an effective amount of ibudilast or a pharmaceutically acceptable salt thereof. In another aspect, the present invention provides a method of alleviating one or more negative effects of anxiety exhibited by a subject suffering from a TBI comprising administering pre-injury to the subject who is at risk for TBI an effective amount of ibudilast or a pharmaceutically acceptable salt thereof. In another aspect, the present invention provides a method of attenuation of glial cell activation in a subject suffering from TBI comprising administering to the subject in need thereof an effective amount of ibudilast or a pharmaceutically acceptable salt thereof.

In another aspect, the present invention provides a method of preventing or reducing a frequency of occurrence of one or more negative effects of post traumatic stress disorder (PTSD) or acute stress disorder (ASD) in a subject suffering from a PTSD or an ASD comprising administering to the subject in need thereof an effective amount of ibudilast or a pharmaceutically acceptable salt thereof. In another aspect, the present invention provides a method of alleviating one or more negative effects of PTSD or ASD in a subject suffering from a PTSD or an ASD comprising administering to the subject in need thereof an effective amount of ibudilast or a pharmaceutically acceptable salt thereof. In another aspect, the present invention provides a method of alleviating one or more negative effects of PTSD or ASD in an individual who is on active duty in a branch of a military or who is a military veteran comprising administering to the individual in need thereof an effect amount of ibudilast or a pharmaceutically acceptable salt thereof.

In one embodiment, the one or more negative effects of TBI, PTSD or ASD include anxiety, stress, hyperexcitability, cognitive or memory impairments, motor deficits, or combinations thereof.

Some aspects of the invention provide methods for treating PTSD and/or ASD in a subject by administering to the subject in need of such a treatment a therapeutically effective amount of a TLR-4 antagonist. There are a wide variety of TLR-4 antagonists known to one skilled in the art. Any TLR-4 antagonist can be used in methods of the invention. However, typically TLR-4 antagonists that are pharmaceutically accepted are used in methods of the invention. Exemplary TLR-4 antagonists that are known to be pharmaceutically useful or acceptable include, but are not limited to, ibudilast, also called AV-411 or MN-166; 2-Methyl-1-[2-(1-methylethyl)pyrazolo[1,5-a]pyridin-3-yl]-1-propanone; 2-Isopropyl-3-isobutyrylpyrazolo[1,5-a]pyridine, E5531 (6-O-{2-deoxy-6-O-methyl-4-O-phosphono-3-O—[(R)-3-Z-dodec-5-endoyl oxydecl]-2-[3-oxo-tetradecanoyl amino]-O-phosphono-D-glucopyranose tetrasodium salt), E5564 (Eritoran™) [-D-glucopyranose,3-O-decyl-2-deoxy-6-O-[2-deoxy-3-O-[(3R)-3-methoxydecyl]-6-O-methyl-2-[[(11Z)-1-oxo-11-octadeceny 1]amino]-4-O-phosphono-D-glucopyranosyl]-2-[(1,3-dioxotetradecyl)amino]-1-(dihydrogen phosphate), tetrasodium salt], TAK-242 (Ethyl (6R)-6-[N-(2-chloro-4-fluorophenyl)sulfamoyl]cyclohex-1-ene-1-carboxylate)), CRX-526 [aminoalkyl-glucosaminide-phosphate], Naloxone [7-allyl-4,5α-epoxy-3,14-dihydroxymorphinan-6-one], Valsartan [S)-3-methyl-2-[N-({4-[2-(2H-1,2,3,4-tetrazol-5-yl)phenyl]phenyl}methyl)pentanamido]butanoic acid], Candesartan [2-ethoxy-1-({4-[2-(2H-1,2,3,4-tetrazol-5-yl)phenyl]phenyl}methyl)-1H-1,3-benzodiazole-6-carboxylic acid], or a pharmaceutically acceptable salt thereof, or a combination thereof.

Other aspects of the invention provide methods for preventing or reducing the risk of developing PTSD, ASD, and/or TBI related negative effects. In order to determine whether an individual is a candidate for preventative treatment of PTSD, ASD, and/or TBI related negative effects, the individual's current life situation can be assessed. If the subject is at risk of exposure to a terrifying event or situation in which grave physical harm (including death, either to the individual or someone else) may occur or be likely to occur, or in which grave physical harm may be threatened, then the subject is a candidate for treatment with methods of the invention in order to prevent ASD, PTSD, and/or TBI related negative effects. Traumatic events that may trigger ASD and PTSD include violent personal assaults, natural or human-caused disasters, accidents, and military combat.

If a subject has experienced such a traumatic event but has not yet exhibited negative effects of ASD, PTSD, and/or TBI, the subject can also be treated with methods of the present invention. Without being bound by any theory, it is believed that methods of the invention modulate or interfere with the process by which memories are formed, reinforced, and/or associated with a emotional and/or physical response.

Often, a subject who has experienced a traumatic event but not yet exhibited negative effects of ASD, PTSD, and/or TBI is treated within a week of exposure to such a traumatic event in order to effectively treat ASD and/or PTSD and prevent some or all of the negative effects associated with ASD, PTSD, and/or TBI from occurring. More often, such a subject is treated within 24, 48, or 72 hours of exposure to the trauma, and even more often the subject is treated immediately following the event, i.e., within 1-6 hours of exposure to the traumatic event.

A subject who has already acquired ASD or PTSD can also be effectively treated with methods of the invention. A subject who has acquired ASD or PTSD and who is therefore in need of treatment with methods of the invention can be identified through the diagnosis of the individual by a skilled clinician, such as a psychologist or psychiatrist. Such a skilled clinician can make a diagnosis of PTSD by following the criteria contained in the DSM-IV, which is well known to one skilled in the art.

If a subject exhibits the appropriate combination of symptoms indicating a diagnosis of PTSD as outlined in DSM-IV, then that subject can be treated with methods of the invention. In order to arrive at a diagnosis of PTSD, the subject's symptoms generally must significantly disrupt normal activities and last for more than one month. Diagnosis of another psychiatric disorder, such as depression, alcohol and drug abuse, or other anxiety disorder, may aid in diagnosis, as approximately 80 percent of patients with PTSD also have at least one other psychiatric disorder.

ASD, PTSD, and/or the negative effects of TBI can be prevented or treated by methods of the present invention. Typically, ibudilast or another TLR-4 antagonist is administered to a subject in a quantity sufficient to treat or prevent the symptoms and/or the underlying etiology associated with ASD, PTSD, and/or TBI in the subject. Methods of the invention can also include administering ibudilast or another TLR-4 antagonist in combination with other agents known to be useful in the treatment of PTSD, such as paroxetine and sertraline, either in physical combination or in combined therapy through the administration of a TLR-4 antagonist and agents in succession (in any order).

In another embodiment, the method further comprises (i.e. in addition to administering ibudilast or a pharmaceutically acceptable salt thereof) administering a glial cell attenuating drug. In another embodiment, the glial cell attenuating drug is minocycline, propentofylline, or pentoxifyline.

Administration of ibudilast or a pharmaceutically acceptable salt thereof (or “ibudilast”) or another TLR-4 antagonist according to the present invention can begin immediately following exposure to a traumatic event, such as TBI, typically within the first week following the traumatic event, and often within the first 24-72 hours. Administration of ibudilast or another TLR-4 antagonist can alternatively begin prior to an anticipated traumatic event (such as impending combat), in order to prevent or reduce the severity of subsequent, TBI related symptoms or negative effects, ASD, and/or PTSD. Ibudilast or the other TLR-4 antagonist can also be administered following a subject's experience of symptoms of TBI, ASD and/or PTSD, such as during either the acute, chronic, or delayed-onset phase. The present invention thus includes the use of ibudilast or another TLR-4 antagonist and/or a pharmaceutical composition comprising such compounds to prevent and/or treat ASD or PTSD.

Various methods of the invention include administering ibudilast or another TLR-4 antagonist or a composition comprising ibudilast or another TLR-4 antagonists to a subject to achieve a desired physiological effect. Typically the subject or patient is an animal, often a mammal, and most often a human. Ibudilast or the other TLR-4 antagonist can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic. In one embodiment, the ibudilast or the pharmaceutically acceptable salt thereof is administered orally, or by subcutaneous, intravenous, or intranasal injection.

In another embodiment, ibudilast or the pharmaceutically acceptable salt thereof is administered once daily, twice daily, thrice daily, once every two days, once every three days, or once a week. In another embodiment, the ibudilast or the pharmaceutically acceptable salt thereof is administered for five days, two weeks, one month, two months, three months, six months, a year, two years, or three or more years.

Ibudilast or the other TLR-4 antagonist can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, ibudilast or the other TLR-4 antagonist may be incorporated with excipient and used in the form of ingestible tablets, buccal tablets, troches, capsules, elixirs, suspensions, syrups, wafers, and the like. Such compositions and preparation can contain at least 0.1% of ibudilast or the other TLR-4 antagonist. The percentage of ibudilast or the other TLR-4 antagonist and preparation can, of course, be varied and can conveniently be between about 1 to about 10% of the weight of the unit. The amount of ibudilast or the other TLR-4 antagonist in such therapeutically useful compositions is such that a suitable dosage will be obtained. Typical compositions or preparations according to the present invention are prepared such that an oral dosage unit form contains from about 1 to about 1000 mg of ibudilast or the other TLR-4 antagonist.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, ibudilast or the other TLR-4 antagonist can be incorporated into sustained-release preparations and formulation.

Ibudilast or the other TLR-4 antagonist can also be administered parenterally. Solutions of ibudilast or the other TLR-4 antagonist or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, e.g., sugars or sodium chloride, are typically included. Prolonged absorption of the injectable compositions of agents delaying absorption, e.g., aluminum monostearate and gelatin, can also be included.

Sterile injectable solutions are typically prepared by incorporating ibudilast or the other TLR-4 antagonist in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The therapeutic TLR-4 antagonist can be administered to a subject alone or in combination with pharmaceutically acceptable carriers, as noted above, the proportion of which is determined by the solubility and chemical nature of TLR-4 antagonist, chosen route of administration and standard pharmaceutical practice.

In another embodiment, the ibudilast or the pharmaceutically acceptable salt thereof is administered in an amount ranging from about 30 to about 300 mg free base equivalent of ibudilast per day, preferably 40 to 120 mg free base equivalent of ibudilast per day in a delayed release oral pharmaceutical composition, and more preferably 80 to 100 mg free base equivalent of ibudilast per day as an oral drug product (capsule) such as Pinatos® or Ketas®.

Generally, the physician will determine the dosage of the TLR-4 antagonist which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and typically from about 10 to about 100 mg/day, or from about 0.1 to about 50 mg/Kg of body weight per day and often from about 0.1 to about 20 mg/Kg of body weight per day and can be administered in several different dosage units. Higher dosages, on the order of about 2× to about 4×, may be required for oral administration.

### EXAMPLES

These examples demonstrates the usefulness of ibudilast (MN166) in vivo in alleviating the negative effects of anxiety-like behavior resulting from TBI. TBI was induced using a clinically relevant closed head injury: lateral fluid percussion injury (LFPI). As TBI is involved in the etiology of PTSD and ASD, these examples demonstrate the usefulness of MN166 in treating negative effects related PTSD and ASD as well.

### Example 1

### Post-Injury Administration of Mn166 Alleviates Anxiety-Like Negative Effects of TBI In Vivo

### Materials and Methods

Twenty-four adult viral-free male Sprague-Dawley rats (275-325 g; Harlan Laboratories, Madison, Wis.) were housed in pairs in temperature (23±3° C.) and light (12:12 light:dark) controlled rooms with ad libitum access to food and water. All procedures were performed in accordance with University of Colorado Institutional Animal Care and Use Committee guidelines for the humane use of laboratory rats in biological research. Rats were randomly assigned to 1 of 4 groups (n=6/group; Sham-operated/vehicle-injected, Sham-operated/ibudilast-injected, LFPI/vehicle-injected, LFPI/ibudilast-injected). All rats were shocked approximately 2 weeks following LFPI to enhance freezing behavior in a novel context (a natural fear reaction in this species). After freezing behavior had been established (1 month post-injury) animals were subjected to a 5-day regimen of ibudilast or vehicle (corn oil) injections (once daily for 5 days). Freezing behavior was again assessed at 2 weeks, 1 month, 2 months, and at 3 months post-injection.

Lateral Fluid Percussion Injury. LFPI rats were anesthetized with halothane (4% induction, 2.0-2.5% maintenance) and mounted in a stereotaxic frame. The lateral fluid percussion injury used in this study utilized a PV820 Pneumatic PicoPump (World Precision Instruments, Inc., Sarasota, Fla.) to deliver standardized pressure pulses of air to a standing column of fluid. A 3.0 mm diameter craniotomy was centered at 3 mm caudal to bregma and 4.0 mm lateral of the sagittal suture, with the exposed dura remaining intact. A female Luer-Loc hub (inside diameter of 3.5 mm) was secured over the craniotomy with cyanoacrylate adhesive. Following hub implantation, the animal was removed from the stereotaxic frame and connected to the LFPI apparatus. The LFPI apparatus delivered a moderate impact force (2.0 atmospheres; 10 ms). The injury cap was then removed, scalp sutured and the rats returned to their home cages for recovery. Sham operated rats underwent identical surgical preparation, but did not receive the brain injury.

Ibudilast administration. Treated rats received a 5-day dosing regimen of once-daily ibudilast injections (10 mg/kg, 1 ml/kg subcutaneously in corn oil). Weight was recorded prior to each dosing.

Tests of motor, vestibular and locomotive performance. Baseline testing of motor, vestibular and locomotive performance in all groups was conducted immediately prior to surgery, and motor function was again evaluated following a 1-week recovery period. These tests included forelimb and hindlimb use to assess locomotion, limb use and limb preference, toe spread to assess gross motor response, placing to assess visual and vestibular function, catalepsy rod test to assess postural support and mobility, bracing to assess postural stability and catalepsy and air righting to assess dynamic vestibular function. Scoring ranged from 0 (severely impaired) to 5 (normal strength and function). The individual test scores were summed and a composite neuro-motor score (0-35) was then generated for each animal.

Behavioral Measures. Freezing in a novel context was used to assess levels of anxiety-like behaviors after administration of a foot shock stressor. The shock apparatus consisted of two chambers placed inside sound-attenuating chests. The floor of each chamber consisted of 18 stainless steel rods (4 mm diameter), spaced 1.5 cm center-to-center and wired to a shock generator and scrambler (Colbourn Instruments, Allentown, Pa.). An automated program delivered a 2-sec/1.5 mA electric shock. Rats were transported in black buckets and shocked immediately upon entry to chambers. Following shock, rats were returned to their home cages. Freezing in a novel context was used as a minor stressor to assess freezing behavior in response to novelty. The novel context consisted of a standard rat cage with one vertically and one horizontally striped wall. No aversive stimuli were introduced in the context and no conditioning occurred. Rats were tested (5 minutes) and the percent of freezing behavior was assessed. Freezing was defined as the absence of movement except for heart beat/respiration, and was recorded in 10 sec intervals.

Statistical Analyses: Results are expressed as mean±SEM. Analyses of all behavioral measures used repeated measures (time point post-injury) ANOVAs, with group assignment as the independent variable, followed by Bonferroni post hoc tests for multiple comparisons. Differences with a p-value of <0.05 were considered significant.

### Results

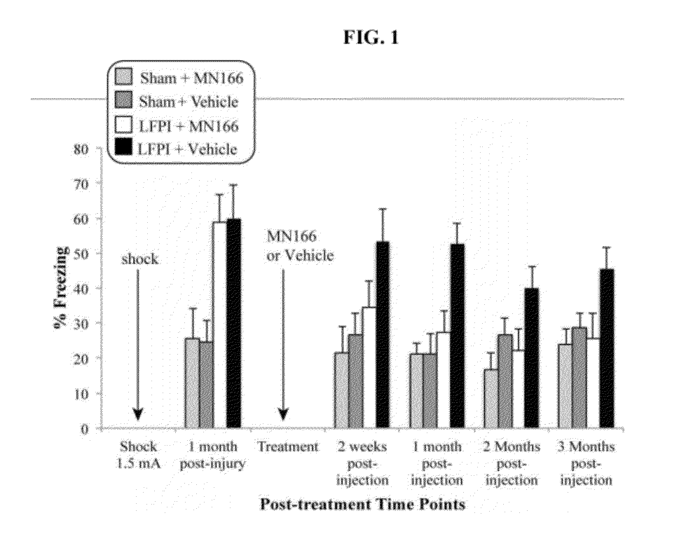

Shocking the rats approximately 2 weeks following LFPI to enhance freezing behavior in a novel context (a natural fear reaction in this species) resulted in LFPI rats freezing approximately 3 times that of controls before ibudilast treatment. See FIG. 1. Ibudilast had no influence on control animals at any subsequent post-injection time-points. In contrast, treated LFPI rats dropped to about 60% freezing compared to untreated injured animals (P<0.05). Even more interestingly, while untreated LFPI rats maintained very high freezing rates up to the 3 month post-treatment time-points recorded, treated LFPI froze substantially less (p<0.01) and were not distinguishable from controls even up to 3 months post-treatment. Ibudilast treatment significantly attenuated the TBI associated increases in freezing behavior, evidenced at the earliest time point and remaining consistent throughout all time points. The results, graphically illustrated in FIG. 1 demonstrate that ibudilast is effective in vivo in alleviating anxiety-like behavior following TBI. There were no significant differences in composite neuro-motor scores between LFPI and control rats, indicating that freezing behavior could not be attributed to motor deficits. It was surprising that administering MN-166 as late as approximately 1 month post-injury alleviated a behavioral readout of PTSD (anxiety-like behavior) with an efficacy of the magnitude observed for the observed duration.

### Example 2

### MN166 can be Prophylactically Effective to Alleviate Anxiety-Like Negative Effects of TBI

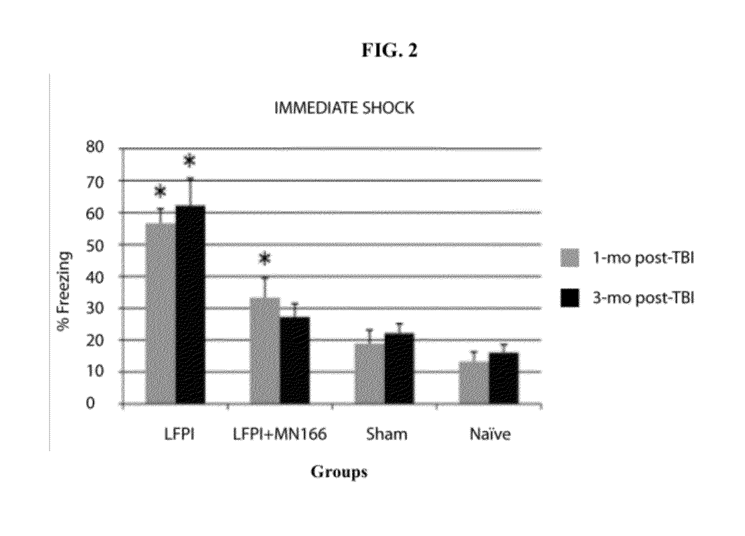

This example demonstrates that administration of MN166 attenuates anxiety-like behavior or post-traumatic anxiety in rats subjected to TBI produced by LFPI. All animals were fear conditioned by receiving shock (1.5 mA; 3-sec) immediately upon being placed in an experimental chamber. Freezing behavior was then measured the following day upon being replaced in the same chamber. Naïve (no surgical procedures) and Sham (LFPI surgery without pressure pulse) froze at a typical rate of approximately 15-20% when measured at 1 and 3 months post-injury (FIG. 2). In contrast, cohorts who received LFPI froze approximately 3 times as long at both time points. Interestingly, in a separate group receiving ibudilast (10 mg/kg in corn oil, 1 ml/kg dose volume) with a 5 day dosing regime (2 days before LFPI, the day of LFPI and 2 days after LFPI), freezing behavior was substantially less than LFPI animals receiving vehicle injections and did not differ significantly from either the Sham or Naïve animals at 3 months post-injury (FIG. 2). This example demonstrates that ibudilast can have preventative effects to reduce anxiety-like behavior in a subject who is at risk of TBI.

### Example 3

### LFPI Animals Demonstrate Anxiety-Like Negative Effects which Effects are Attenuated by MN166

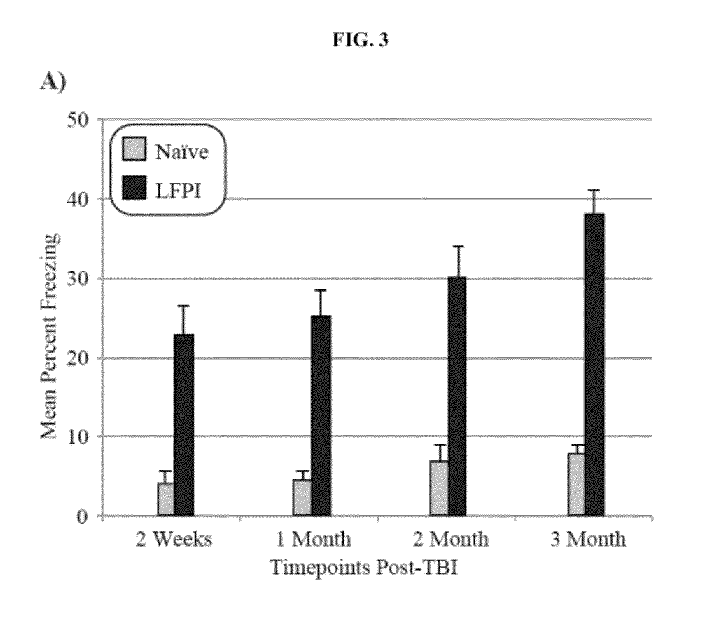

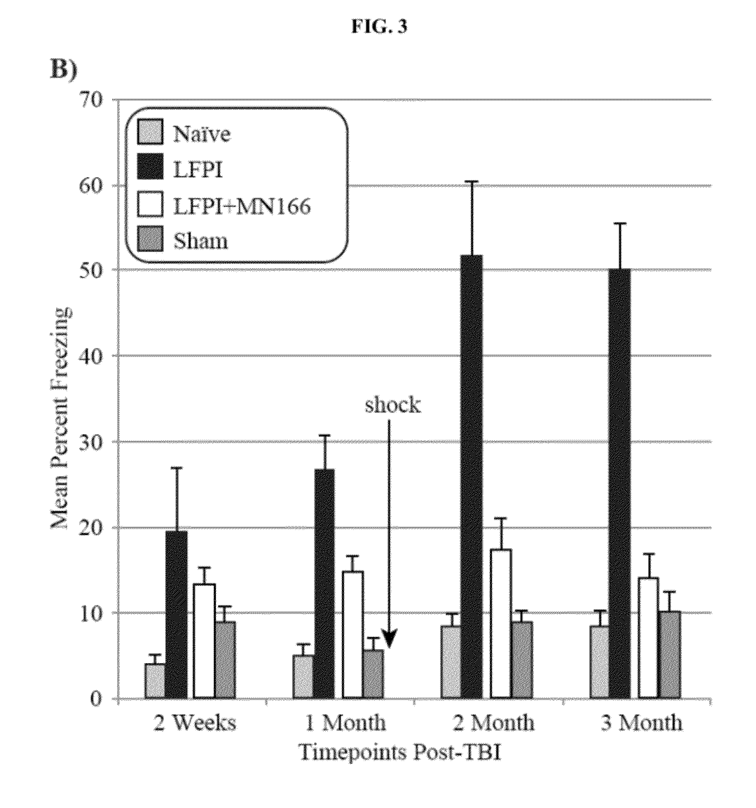

This example illustrates the usefulness of LFPI animals for demonstrating anxiety-like behavior and the effect of MN166 to attenuate such anxiety like behavior. Freezing behavior in rats was elicited substantially in the manner described before. When freezing behavior was made the dependent variable after placement in a novel context, large and stable differences between Naïve and LFPI animals were seen (FIG. 3). LFPI animals froze approximately 20-40% of the time compared to 5-10% for Naïve animals (FIG. 3A). Increased freezing behavior in LFPI compared to Naïve animals was significant at all time points (p<0.001).

FIG. 3B shows freezing behavior in a novel context for a separate set of animals that received shock at 1 month post-injury. These animals included Naïve and LFPI groups as in FIG. 3A, but also LFPI+MN166 (Animals received a 5-day dosing regimen in which they received once-daily ibudilast injections 24 hr prior to LFPI, the day of surgery and LFPI, and 3 days following LFPI (10 mg/kg, 1 ml/kg subcutaneously in corn oil)) and a Sham surgery group. Overall, LFPI animals froze substantially more than Naïve, LFPI+MN166, and Sham groups at the 1, 2 and 3 month time points (p<0.001). Similar to the animals of FIG. 3A, at 2 weeks and 1 month post-injury, LFPI animals froze approximately 20-30% of the time in the novel context compared to approximately 5% for Naïve animals (p<0.001). Shock (FIG. 3B; arrow) increased freezing behavior in the LFPI animals to approximately 50% at the 2 and 3 month time points, substantially higher than Naïve animals (p<0.001), who did not increase substantially. Thus, while exposure to a novel context resulted in enhanced freezing behavior at 2 and 3 months post-injury (FIG. 3A), the added stressor of shock significantly increased freezing at these time points (FIG. 3B; p<0.001). All effects appear to be due to the LFPI itself since animals with Sham surgery did not differ significantly from Naïve animals at any of the time points.

In contrast, LFPI animals treated with MN166 (FIG. 3B) froze more than Naïve and Sham animals at 2 week and 1 month time points before shock (p<0.015 and 0.002, respectively), but were not distinguishable from Naïve or Sham animals at 2 and 3 months post-injury (FIG. 3B; p=0.060 and 0.336). While MN166 treated animals generally froze more than Naïve and Sham controls, a striking effect of treatment was greatly reduced freezing compared to untreated LFPI animals, reaching significance at 1, 2 and 3 months post-injury (p<0.01, 0.001 and 0.0001, respectively). At 3 months post-injury, when MN166 treated animals froze at 14% and could not be distinguished from Naïve (8%) or Sham (10%) controls, untreated LFPI animals froze 50% of the time.

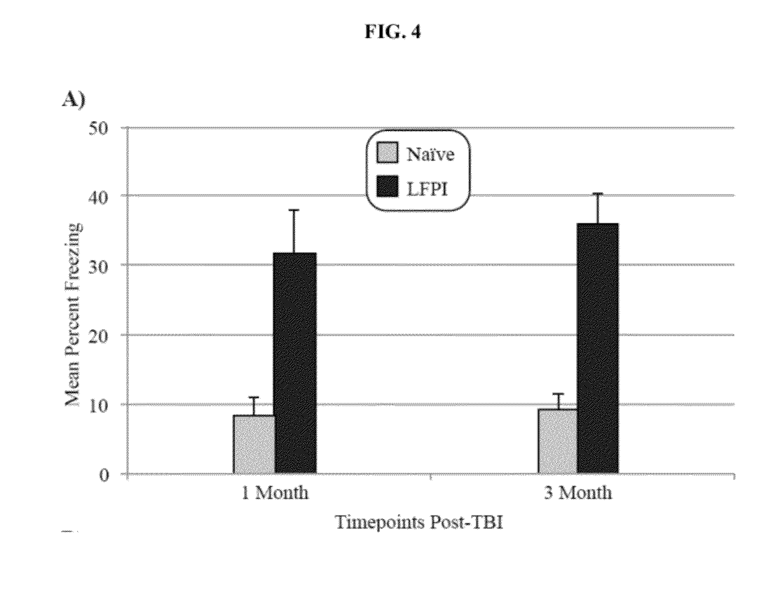

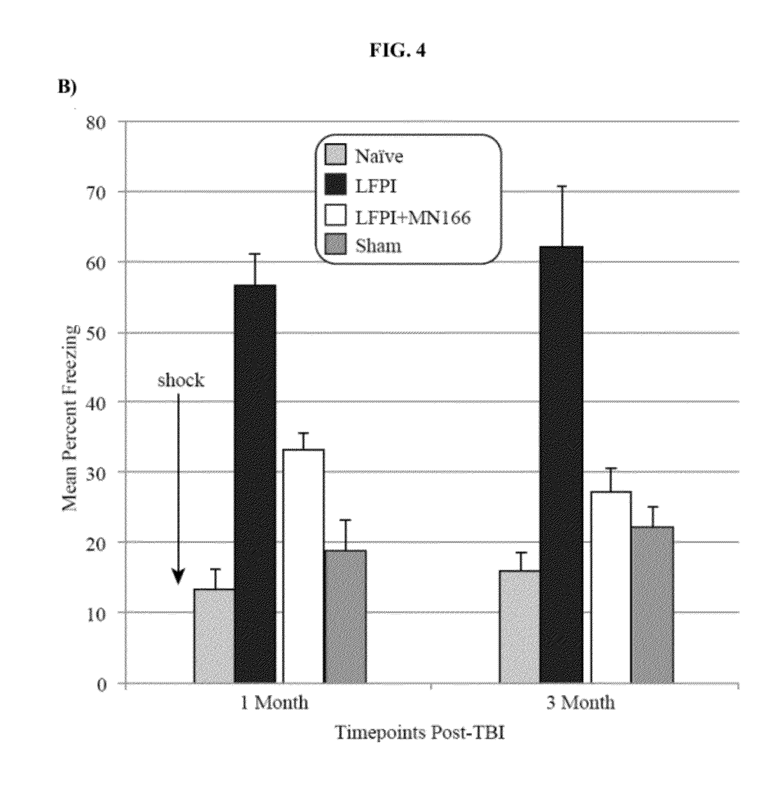

FIG. 4 shows freezing behavior of animals in the absence of immediate shock (FIG. 4A) and a separate group receiving shock (FIG. 4B). Freezing behavior of animals that did not receive shock was very similar to their performance in a novel environment. Animals with LFPI froze approximately 3-4 times longer (30-40%) than Naïve animals (<10%) when placed in the test chamber (FIG. 4A). As with novel context, freezing behavior in LFPI animals differed significantly from Naïve animals at the 1 and 3-month post-injury time points (p<0.001 and 0.0001). In Naïve animals receiving immediate shock (FIG. 4B; shock delivered 24 hours before testing, 1 month post-injury), freezing behavior was not significantly different than Naïve animals that did not receive shock at both 1 and 3 months post-injury (FIG. 4A, p=0.934 and 0.889, respectively). However, LFPI animals froze substantially more (approximately 60%) after shock compared to LFPI animals receiving no shock (30-35%; p<0.001 and 0.003, respectively). They also froze significantly more at 1 and 2 months post-injury than similarly shocked Naïve (10-15%; p<0.0001 and 0.0001, respectively) and Sham (15-20%; p<0.0001 and 0.0001, respectively) animals. LFPI animals consistently froze more than all other groups, at all time points, with or without shock. Again, Sham and Naïve did not differ at any time point post-injury. As in the novel environment, MN166 treatment significantly reduced freezing (25-35%) in LFPI animals (FIG. 4B) compared to the untreated LFPI animals (p<0.003 and 0.0001, respectively). While treated LFPI animals did not significantly differ from Sham animals at either time point (p<0.101 and 0.889, respectively), they did freeze more than Naïve controls (p<0.01) at 1 month. However, by 3 months this difference was no longer significant (p<0.493).

### CONCLUSION

A negative effect of TBI, an anxiety-like freezing behavior, was induced in vivo following LFPI and optionally with shocking and placing the subject in a novel context. Administration of MN166 to subjects, at various time points relative to the traumatic injury suffered by the subjects, alleviated the negative effects of anxiety-like behavior resulting from the traumatic injury.

What is claimed is:

1. A method of alleviating one or more negative effects of anxiety exhibited by a subject suffering from a traumatic brain injury (TBI) comprising administering to the subject in need thereof an effective amount of ibudilast or a pharmaceutically acceptable salt thereof.

2. A method of inhibiting an onset of one or more negative effects of traumatic brain injury (TBI) exhibited by a subject suffering from TBI comprising administering to the subject in need thereof an effective amount of ibudilast or a pharmaceutically acceptable salt thereof.

3. The method of claim 1 in which the one or more negative effects of TBI include anxiety, stress, hyperexcitability, cognitive or memory impairments, motor deficits, or combinations thereof.

4. A method of preventing or reducing a frequency of occurrence of one or more negative effects of post traumatic stress disorder (PTSD) or acute stress disorder (ASD) in a subject suffering from a PTSD or an ASD comprising administering to the subject in need thereof an effective amount of ibudilast or a pharmaceutically acceptable salt thereof.

5. The method of claim 4 in which the one or more negative effects of PTSD or ASD include anxiety, stress, hyperexcitability, cognitive or memory impairments, motor deficits, or combinations thereof.

6. A method of alleviating one or more negative effects of post traumatic stress disorder (PTSD) or acute stress disorder (ASD) in a subject suffering from a PTSD or an ASD comprising administering to the subject in need thereof an effective amount of ibudilast or a pharmaceutically acceptable salt thereof.

7. The method of claim 6 in which the one or more negative effects of PTSD or ASD include anxiety, stress, hyperexcitability, cognitive or memory impairments, motor deficits, or combinations thereof.

8. The method of claim 1 in which the ibudilast or the pharmaceutically acceptable salt thereof is administered in an amount ranging from about 30 to about 300 mg free base equivalent of ibudilast per day.

9. The method of claim 1 in which the ibudilast or the pharmaceutically acceptable salt thereof is administered orally, or by subcutaneous, intravenous, or intranasal injection.

10. The method of claim 1 in which the ibudilast or the pharmaceutically acceptable salt thereof is administered once daily, twice daily, thrice daily, once every two days, once every three days, or once a week.

11. The method of claim 1 in which the ibudilast or the pharmaceutically acceptable salt thereof is administered for five days, two weeks, one month, two months, three months, six months, a year, two years, or three or more years.

12. A method of alleviating one or more negative effects of anxiety exhibited by a subject suffering from a traumatic brain injury (TBI) comprising administering pre-injury to the subject who is at risk for TBI an effective amount of ibudilast or a pharmaceutically acceptable salt thereof.

13. The method of claim 12 in which the method further comprises administering post-injury to the subject an effective amount of ibudilast or a pharmaceutically acceptable salt thereof.

14. The method of claim 1, further comprising administering a glial cell attenuating drug that is minocycline, propentofylline, or pentoxifyline.

15. A method of alleviating one or more negative effects of post-traumatic stress disorder or acute stress disorder in an individual who is on active duty in a branch of a military or who is a military veteran comprising administering to the individual in need thereof an effect amount of ibudilast or a pharmaceutically acceptable salt thereof.

16. A method for treating post-traumatic stress disorder or acute stress disorder in a subject comprising administering to the subject in need of such a treatment an effective amount of a TLR-4 antagonist.

17. The method of claim 16 in which the TLR-4 antagonist comprises Ibudilast also called AV-411 or MN-166 (3-Isobutyryl-2-isopropylpyrazolo[1,5-a]pyridine; 2-Methyl-1-[2-(1-methylethyl)pyrazolo[1,5-a]pyridin-3-yl]-1-propanone; 2-Isopropyl-3-isobutyrylpyrazolo[1,5-a]pyridine, E5531 (6-O-{2-deoxy-6-O-methyl-4-O-phosphono-3-O—[(R)-3-Z-dodec-5-endoyl oxydecl]-2-[3-oxo-tetradecanoyl amino]-O-phosphono-D-glucopyranose tetrasodium salt), E5564 (Eritoran™) [-D-glucopyranose,3-O-decyl-2-deoxy-6-O-[2-deoxy-3-O-[(3R)-3-methoxydecyl]-6-O-methyl-2-[[(11Z)-1-oxo-11-octadeceny 1]amino]-4-O-phosphono-D-glucopyranosyl]-2-[(1,3-dioxotetradecyl)amino]-1-(dihydrogen phosphate), tetrasodium salt], TAK-242 (Ethyl (6R)-6-[N-(2-chloro-4-fluorophenyl)sulfamoyl]cyclohex-1-ene-1-carboxylate)), CRX-526 [aminoalkyl-glucosaminide-phosphate], Naloxone [7-allyl-4,5α-epoxy-3,14-dihydroxymorphinan-6-one], Valsartan [S)-3-methyl-2-[N-({4-[2-(2H-1,2,3,4-tetrazol-5-yl)phenyl]phenyl}methyl)pentanamido]butanoic acid], Candesartan [2-ethoxy-1-({4-[2-(2H-1,2,3,4-tetrazol-5-yl)phenyl]phenyl}methyl)-1H-1,3-benzodiazole-6-carboxylic acid], or a combination thereof.

18. The method of claim 16 in which the TLR-4 antagonist is administered parenterally, orally, nasally, buccally, intravenously, intramuscularly, subcutaneously, intrathecally, epidurally, transdermally, intracerebroventricularly, by osmotic pump, by inhalation, or combinations thereof.

19. The method of claim 16 in which the TLR-4 antagonists is administered at least once daily.

20. The method of claim 16 in which the amount of TLR-4 antagonists administered is at least about 7.5-10 mg/day/Kg.

21. A method for preventing or reducing the frequency of post-traumatic stress disorder occurrence in a subject comprising administering to the subject in need of such a treatment a therapeutically effective amount of a TLR-4 antagonist.
